(19) United States
(12) Patent Application Publication
(10) Pub. No.: US 2009/0093037 A1
(43) Pub. Date: Apr. 9, 2009

(54) METHOD AND APPARATUS FOR ACCELERATING GROWTH OF LACTIC ACID BACTERIA WITH ELECTRIC FIELD

(75) Inventors: MinSun KIM, Seoul (KR); JEONGJE PARK, Gyeonggi-do (KR); JAECHAN PARK, Gyeonggi-do (KR); KYUNGIL CHO, Seoul (KR)

Correspondence Address: CANTOR COLBURN, LLP, 20 Church Street, 22nd Floor, Hartford, CT, 06103, US

(73) Assignee: SAMSUNG ELECTRONICS, CO., LTD., Gyeonggi-do (KR)

(21) Appl. No.: 12/030,581

(22) Filed: Feb. 13, 2008

(30) Foreign Application Priority Data
Oct. 4, 2007 (KR) 10-2007-0099918

(51) Int. Cl. C12N 13/00; C12M 3/00

(52) U.S. Cl. 435/173.8; 435/289.1

(57) ABSTRACT

The method and apparatus for accelerating growth of lactic acid bacteria, which apply an electric field to the lactic acid bacteria using an electric field in a frequency band of 1 kHz to 1 MHz, can be applied to all lactic acid bacteria used in home and industries to accelerate the growth of them and can be used to increase the number of the lactic acid bacteria and the production of all metabolites (for example, lactic acid and carbon dioxide) of the lactic acid bacteria. The invention increases the lactic acid bacteria and the production of the metabolites of the lactic acid bacteria, thereby activating the beneficial fermentation action of the processed foods such as fermented milk, lactic acid bacteria-containing beverage, toenjang or salted foods (kimchi or pickle and the like). In addition, the invention lowers pH of the foods to suppress the proliferation of the harmful microbes, and makes the low oxygen/oxygen-free environment using the carbon dioxides produced as metabolism by-products, thereby increasing the flavor of the foods and prolonging the preservation period.

### CROSS-REFERENCE TO RELATED APPLICATIONS

This application claims all benefits of Korean Patent Application No. 10-2007-0099918 filed on Oct. 4, 2007 in the Korean Intellectual Property Office, the disclosures of which are incorporated herein by reference.

### BACKGROUND OF THE INVENTION

1. Field of the Invention

The present invention relates to a method and an apparatus for accelerating growth of lactic acid bacteria by applying an electric field to the lactic acid bacteria with a non-contact electric field using alternating current electric field having an intensity of 10V˜1 MV/m in a frequency band of 1 kHz to 1 MHz or direct current electric field having the same intensity, and more particularly to a method that can be applied to all lactic acid bacteria used in home and industries and can be used to increase the lactic acid bacteria and the production of all metabolites (for example, lactic acid and carbon dioxide) of the lactic acid bacteria and to promote the concomitant beneficial effect.

2. Description of the Related Art

The bacteria group referred to as lactic acid bacteria is a general term of bacteria that ferment saccharide at oxygen-free state to produce a large amount of lactic acid. The fermentation means that the lactic acid bacteria produce the lactic acid from sugar, which can be classified into homofermentative and heterofermentative. The homofermentative is fermentation that produces mainly the lactic acid from glucose by 85% or more and the heterofermentative is fermentation that produces the lactic acid and the other by-products such as volatile materials, alcohol and the like from glucose.

The fermentation of lactic acid bacteria is used in a variety of industrial fields, for example fermented milk, lactic acid bacteria beverage, dairy products (yogurt, cheese, fermented butter), brewed products (refined rice wine, toenjang, soy sauce, wine), kimchi or sauerkraut, salted foods such as pickles, sausage, fermented soybean milk or other health foods.

The acceleration of growth of the lactic acid bacteria has been conventionally performed while maintaining the optimal temperature condition of about 37° C. and supplying the nutrient ingredients. However, regarding the production and maturation of the foods using the lactic acid bacteria, the above temperature condition has a bad influence on the taste and quality.

### SUMMARY OF THE INVENTION

The inventors found that when the lactic acid bacteria are applied with alternating current electric field having an intensity of 10V˜1 MV/m in a frequency band of 1 kHz to 1 MHz or direct current electric field having the same intensity, the growth of the lactic acid bacteria is accelerated even at the low temperatures. Accordingly, an object of the invention is to provide a method and an apparatus for accelerating growth of lactic acid bacteria, in which an electric field is applied to the lactic acid bacteria so as to increase production yields of the lactic acid bacteria and metabolites of the lactic acid bacteria.

There is provided a method for accelerating growth of lactic acid bacteria comprising a step of applying an electric field so that alternating current electric field having an intensity of 10V˜1 MV/m in a frequency band of 1 kHz to 1 MHz or direct current electric field having the same intensity is applied to the lactic acid bacteria.

In addition, there is provided an apparatus for accelerating growth of lactic acid bacteria comprising a receptacle made of insulating material, two electrodes attached on outer surfaces of a bottom and a cover of the receptacle and an electric field applying device connected to the two electrodes wherein the electric field applying device applies alternating current electric field having an intensity of 10V˜1 MV/m in a frequency band of 1 kHz to 1 MHz or direct current electric field having the same intensity.

By applying the electric field to the lactic acid bacteria, the growth of lactic acid bacteria is accelerated even at low temperatures to increase the production of the lactic acid bacteria and to promote the fermentation. In addition, pH is lowered due to the fermentation, i.e., the acidic environment is made to suppress the proliferation of the other harmful microbes. Moreover, the low oxygen/oxygen-free environment using the metabolites (for example, lactic acid and carbon dioxide) of the lactic acid bacteria is made to increase the flavor of the foods and to prolong the preservation period.

### BRIEF DESCRIPTION OF THE DRAWINGS

The above and other objects, features and advantages of the present invention will be more apparent from the following detailed description taken in conjunction with the accompanying drawings, in which:

FIG. 1 schematically shows an apparatus for accelerating growth of lactic acid bacteria according to an embodiment of the invention;

FIG. 2 is a graph of optical density showing lactic acid bacteria proliferation after electric fields of 10 Hz, 100 Hz, 1 kHz, 10 kHz, 100 kHz and 1 MHz are applied;

FIG. 3 is a photograph showing the number of smear bacteria observed with a microscope, which are exhibited in an agar plate after 5 days from the electric field application to Leuconostoc mesenteroides; and

FIG. 4 is a graph comparing the ATP values before and after the promotion of lactic acid bacteria growth according to an embodiment of the invention.

### DESCRIPTION OF REFERENCE MARKS FOR THE MAIN PORTIONS OF THE DRAWINGS

1: receptacle

2: electrode

3: electric field applying device

### DETAILED DESCRIPTION OF THE PREFERRED EMBODIMENTS

Hereinafter, a preferred embodiment of the present invention will be described with reference to the accompanying drawings.

The invention provides a method for accelerating growth of lactic acid bacteria in which an electric field is applied to the lactic acid bacteria using alternating current electric field having an intensity of 10V˜1 MV/m in a frequency band of 1 kHz to 1 MHz or direct current electric field having the same intensity. The invention can be applied to all lactic acid bacteria used in home and industries and can be used to promote the growth of the lactic acid bacteria, to increase the number of lactic acid bacteria and to produce all metabolites produced from the lactic acid bacteria.

A principle in which the growth of lactic acid bacteria is accelerated due to the application of electric field according to the invention is as follows. All cells including the lactic acid bacteria require the energy to synthesize ingredients necessary for the growth and proliferation of the cells and the cloning of DNA. The energy is generated in a form of ATP and stored in the cell. When the energy does not reach a predetermined content, the life activity is difficult. There are two methods of synthesizing the ATP in the cell. One is to decompose the nutrient ingredients such as glucose and to convert them into ATP through the breath process. The other is to accumulate the hydrogen ions (H+), i.e., protons at an outside of the cell membrane and to produce the ATP from ADP while passing the protons through ATPase (ATP decomposition enzyme) present on the cell membrane when there occurs a predetermined level of gradient between in and out of the cell membrane. Therefore, the passing of the charged particles such as protons is accelerated due to an electric field, and when the generation of ATP is increased, the growth rate of the cell is faster as long as there are the ingredients necessary for the cell growth.

According to an embodiment of the invention, alternating current electric field having an intensity of 10V˜1 MV/m in a frequency band of 1 kHz to 1 MHz or direct current electric field having the same intensity is applied. When alternating current electric field having a frequency out of the above range is applied, an expected effect may be not exhibited or the growth of the lactic acid bacteria may be decreased. In addition, when the electric field having intensity less than 10V/m is applied, the effect is negligible or may be not exhibited. When the intensity is greater than IMV/m, a problem of safety is caused, so that it may be difficult to actually produce a product.

According to an embodiment of the invention, when the alternating current electric field is applied, a waveform is not particularly limited. For example, a sine or square wave may be used.

The main lactic acid bacteria used in an embodiment of the invention can be classified into 6 (six) geneses depending on bacteria types, fermentation aspects and the like, i.e., Lactobacillus genus of a rod shape, Streptococcus genus of a round shape, Bifidobacterium genus, Leuconostoc genus, Pediococcus genus and Lactococcus genus.

The Lactobacillus genus is a compound word of Lacto meaning lactose of milk and bacillus meaning a rod shape, and means the rod-shaped bacillus that decomposes the lactose in the milk. Compared to the other lactic acid bacteria, the lactic acid bacteria are most used scientifically and industrially, and include bulgaricus, acidophilus and the like. More specifically, there are Lactobacillus acidophilus, Lactobacillus lactis, Lactobacillus delbrueckii, Lactobacillus sleichmannii, Lactobacillzis salivarius, Lactobacillus bavaricus, Lactobacillus casei, Lactobacillus coryniformis, Lactobacillus curvatus, Lactobacillus plantarum, Lactobacillus sake, Lactobacillus brevis, Lactobacillus buchneri, Lactobacillus cellobiosus, Lactobacillus confusus, Lactobacillus coprophilus, Lactobacillus fermentatum, Lactobacillus sanfrancisco and the like.

The Streptococcus genus is a compound word of strepto (chain) meaning a chain-shaped connection and coccus (round) meaning a round shape, and means that ‘round bacilli are connected in a chain form.’ The representative bacilli include Streptococcus thermophilus, Streptococcus bovis and the like.

The Bifidobacterium genus is bacillus, produces the lactic acid and the acetic acid to perform an intestinal beneficial action and prevails in the intestines of a breast-fed child. Bifido means taking branches (i.e., Y or V shape) and it includes bifidus and the like.

The Leuconostoc genus is diplococcus in which two bacterial cells are respectively connected, is mainly found in kimchi and cheese and is little used in fermented milk.

The Pediococcus genus is streptococcus in which four bacterial cells are respectively connected and is mainly found in kimchi or salted foods, and the lactic acid bacteria belonging to the genus are not also used in the fermented milk, as the Leuconostoc genus. The representative lactic acid bacteria in this genus include Pediococcus acidilactici, Pedicoccus damnosus, Pediococcus pentococcus and the like.

The Lactococcus genus was classified into streptococcus up to recently but is classified into a new genus now, is mainly found in cheese, fermented butter and the like. The lactic acid bacterial belonging to the genus is found to include the lactic acid bacteria that produce the antibacterial materials. The representative lactic acid bacteria in this genus include Leuconostoc dextranicum, Leuconostoc mesenteroides, Leuconostoc paramesenteroides and the like.

Furthermore, there are Enterococcus faeciuni, Enterococcus faecalis and the like.

In an apparatus for accelerating growth of lactic acid bacteria according to an embodiment of the invention, a variety of electrodes, for example a metal electrode, a transparent electrode or an electrode using a plastic conductive material may be used as an electrode. Further, the electrodes are connected to an electric field applying device so that one is grounded and the other is applied with voltage.

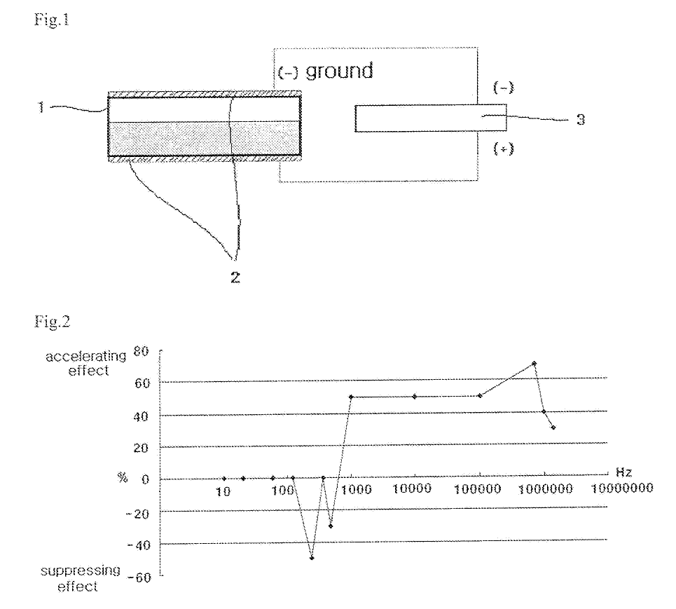

According to an embodiment of the invention, the electric field is applied such a non-contact manner that the electric field is applied while the electrodes are not contacted to the lactic acid bacteria or the lactic acid bacteria-mixed solution. By using the non-contact manner, the method for accelerating growth of lactic acid bacteria can be applied to various type of foods or lactic acid bacteria-mixed solution. FIG. 1 schematically shows an apparatus for accelerating growth of lactic acid bacteria, in which the electric field is applied in a non-contact manner. In order to apply the electric field in a non-contact manner, the lactic acid bacteria or lactic acid bacteria-mixed solution is put in a receptacle made of insulating material. Then, the receptacle is located between two electrodes which are connected to an electric field applying device. At this time, a receptacle is used which is designed so that the two electrodes 2 made of conductive material are attached to on outer surfaces of a bottom and a cover of the receptacle I made of insulating material, and the electrodes are connected to the electric field applying device 3 to perform the non-contact electric field application.

Hereinafter, the structure of the invention will be more specifically described with reference to the embodiments. However, it should be noted that the below examples are provided to illustrate the invention, not to limit it.

### Embodiments

Firstly, the Leuconostoc mesenteroides, which are kimchi lactic acid bacteria, were cultured in dedicated culturing solution MRS medium (Gibco) in a concentration of 10⁶ CFU/ml. The mixture solution of the lactic acid bacteria and the culturing solution was put in 6-well plates made of polypropylene (insulating material), to which the alternating current electric field of sine wave having a frequency of 10 Hz, 100 Hz, 1 kHz, 10 kHz, 100 kHz and 1 MHz was then applied in an intensity of 100 V/cm while the plates were not directly contacted. to the electrodes for applying the electric field as shown in FIG. 1. The proliferation degree was assayed with optical density at 600 nm of the mixture solution of the lactic acid bacteria and the culturing solution.

As shown in FIG. 2 showing the result, when the electric field was applied in the frequency of 1 kHz to 1 MHz, the growth of lactic acid bacteria was accelerated. Compared to the case where the electric. field was not applied (control group), the growth of lactic acid bacteria was more accelerated by about 130%. The effect of the growth acceleration may be different depending on the temperature, the time and the concentration of the lactic acid bacteria.

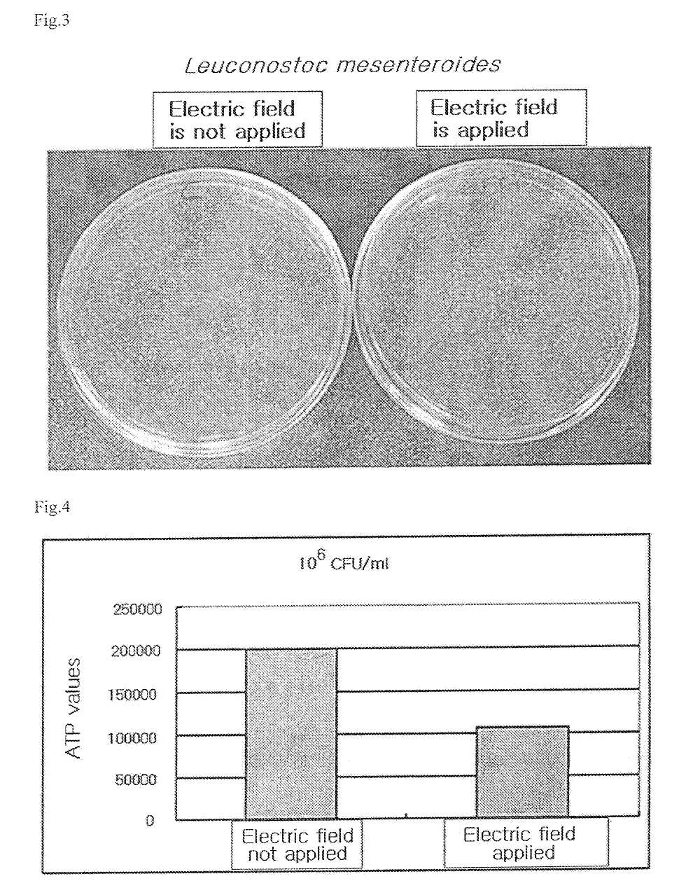

FIG. 3 is a photograph showing the number of smear bacteria observed with a microscope, which are exhibited in an agar plate after 5 days from the electric field application in the above embodiment. In case of the electric field application, it could be seen that the growth of lactic acid bacteria was accelerated.

FIG. 4 is a graph comparing the ATP values before and after the promotion of lactic acid bacteria growth according to an embodiment of the invention. When the electric field was applied, it could be seen that the ATP was more generated.

According to the invention, when the alternating current electric field having an intensity of 10V˜1 MV/m in a frequency band of 1 kHz to 1 MHz or direct current electric field having the same intensity is applied to the lactic acid bacteria, and the growth of the lactic acid bacteria is activated to increase the production of the lactic acid bacteria and the other metabolites, thereby improving the beneficial fermentation action of the foods.

While the invention has been shown and described with reference to certain preferred embodiments thereof, it will be understood by those skilled in the art that various changes in form and details may be made thereto without departing from the spirit and scope of the invention as defined by the appended claims.

What is claimed is:

1. A method for accelerating growth of lactic acid bacteria comprising applying alternating current electric field having an intensity of 10V˜1 MV/m in a frequency band of 1 kHz to 1 MHz or direct current electric field having the same intensity to the lactic acid bacteria.

2. The method according to claim 1, wherein the lactic acid bacteria are Lactobacillus genus, Streptococcus genus, Bifidobacterium genus, Leuconostoc genus, Pediococcus genus or Lactococcus genus.

3. An apparatus for accelerating growth of lactic acid bacteria comprising:
a receptacle 1 made of insulating material;
two electrodes 2 attached on outer surfaces of a bottom and a cover of the receptacle; and
an electric field applying device 3 connected to the two electrodes
wherein the electric field applying device applies alternating current electric field having an intensity of 10V˜1 MV/m in a frequency band of 1 kHz to 1 MHz or direct current electric field having the same intensity.

4. The apparatus according to claim 3, wherein one of the two electrodes is grounded and the other is connected to an anode or a cathode of the electric field applying device.

5. The apparatus according to claim 3, wherein the receptacle 1 includes lactic acid bacteria therein and the lactic acid bacteria are Lactobacillus genus, Streptococcus genus, Bifidobacterium genus, Leuconostoc genus, Pediococcus genus or Lactococcus genus.
